(19) United States
(12) Patent Application Publication
(10) Pub. No.: US 2024/0173233 A1
(43) Pub. Date: May 30, 2024

(54) COSMETIC SACHET FOR REDUCING AGE-RELATED SYMPTOMS IN HUMAN SKIN

(71) Applicant: Bloo Pharmapeutica Inc., Vancouver (CA)

(72) Inventors: Susan Quakenbush, Victoria (CA); Ken Lomas, Vancouver (CA)

(73) Assignee: Bloo Pharmapeutica Inc., Vancouver (CA)

(21) Appl. No.: 18/522,045

(22) Filed: Nov. 28, 2023

(30) Foreign Application Priority Data
Nov. 28, 2022 (CA) 3183356

(51) Int. Cl. A61K 8/49; A61K 8/02; A61K 8/19; A61K 8/365; A61K 8/73; A61K 8/92; A61Q 19/08

(52) U.S. Cl. CPC A61K 8/49; A61K 8/0204; A61K 8/19; A61K 8/365; A61K 8/732; A61K 8/922; A61Q 19/08; A61K 2800/522

(57) ABSTRACT

A cosmetic sachet is provided for reducing age-related symptoms in human skin, the cosmetic sachet comprising: a bag comprising a water dispersing paper; and a cosmetic composition, the cosmetic composition releasably retained in the bag and comprising sodium bicarbonate, citric acid, magnesium sulfate heptahydrate, starch, highly saturated plant oil and methylene blue.

### CROSS REFERENCE TO RELATED APPLICATIONS

This application claims benefit of Canadian Patent Application Serial No. 3,183,356, filed on Nov. 28, 2022, entitled COSMETIC SACHET FOR REDUCING AGE-RELATED SYMPTOMS IN HUMAN SKIN, the contents of which are incorporated herein by reference.

### FIELD

The present technology is a cosmetic sachet containing a cosmetic composition for decreasing aging related changes to human skin. More specifically, it is a cosmetic sachet comprising a water dispersible bag containing a cosmetic composition which includes methylene blue.

### BACKGROUND

Large sums of money have been spent on researching and developing cosmetic compositions for reducing or masking aging-related changes to human skin. Some research has pointed to the use of methylene blue as an anti-oxidant for reducing skin aging. For example, it is disclosed in “Anti-Aging Potentials of Methylene Blue for Human Skin Longevity” (Zheng-Mei Xiong, Mike O'Donovan, Linlin Sun, Ji Young Choi, Margaret Ren & Kan Cao Scientific Reports volume 7, Article number: 2475 (2017)) that oxidative stress is the major cause of skin aging that includes wrinkles, pigmentation, and weakened wound healing ability. Application of antioxidants in skin care is well accepted as an effective approach to delay the skin aging process. Methylene blue (MB), a traditional mitochondrial-targeting antioxidant, showed a potent reactive oxygen species (ROS) scavenging efficacy in cultured human skin fibroblasts derived from healthy donors and from patients with progeria, a genetic premature aging disease. In comparison with other widely used general and mitochondrial-targeting antioxidants, we found that MB was more effective in stimulating skin fibroblast proliferation and delaying cellular senescence. The skin irritation test, performed on an in vitro reconstructed 3D human skin model, indicated that MB was safe for long-term use, and did not cause irritation even at high concentrations. Application of MB to this 3D skin model further demonstrated that MB improved skin viability, promoted wound healing and increased skin hydration and dermis thickness. Gene expression analysis showed that MB treatment altered the expression of a subset of extracellular matrix proteins in the skin, including upregulation of elastin and collagen 2A1, two essential components for healthy skin. Altogether, our study suggests that MB has a great potential for skin care.

Russian Patent Application Publication No. 0002774769 discloses that a step-by-step study of existing studies was carried out and it was found that [7-(dimethylamino)phenothiazine-3-ylidene]-dimethylazanium chloride (methylene blue) at a concentration of 0.1·10-6 M to 1.0·10-6 M has a marked biological activity, an antioxidant effect in cell culture tests, and a well-known antiseptic effect. The composition for use in a cosmetic product applied to the skin includes, in wt. %: methylene blue 0.00001-0.001, N-acetyl-L-cysteine 0.00002-15, excipients 0-9.5, water 84.999-99.99997.

U.S. patent Ser. No. 10/603,325 discloses a method of alleviating age-related symptoms in a mammal, which comprises a step of administering to a mammal in need thereof, an effective amount of a composition containing an effective amount of at least methylene blue.

United States Patent Application Publication No. 20220064034 discloses a submersible vessel which can act as a reusable bath bomb delivery system is provided. The vessel may include a housing and an opening port. The interior of the housing and ports form a chamber, which can be segmented into multiple sub-chambers. Inlet one-way valve(s) and outlet one-way valve(s) are provided on the vessel. To use, the chamber is partially filled with a mixture including an effervescent material. When water is introduced through the inlet valve, it reacts with the effervescent material, releasing carbon dioxide and dissolving the other ingredients in the mixture, building pressure in the chamber. When the pressure builds to a certain level, the inlet valve(s) are closed, and the outlet valve(s) are opened. The outlet valves may be positioned such that when the fluid and gas is expelled, the force generated may cause the vessel to spin or be propelled in the bath.

U.S. Pat. No. 8,877,678 discloses a water dispersion paper having quick water dispersion property and high strength (printability) and whose paper surface pH is 6 to 8 (neutral range), and also to provide a water dispersion coated paper designed to ensure quick water dispersion property and prevent discoloration over time.

United States Patent Application 20210032002 discloses a packaging film constructed from water-dispersible and/or biodegradable compositions. Particularly, the disclosed film comprises a first layer constructed from one or more water-dispersible materials, such as water-dispersible paper. The film further comprises a second layer constructed from one or more biodegradable materials, such as poly(hydroxyalkanoate). The first and second layers can be constructed to form a packaging material used to enclose a wide variety of products, including liquids and moisture-sensitive solids. Advantageously, the disclosed film (and associated packaging materials) is dissolvable in water and/or biodegrade when exposed to landfill conditions and/or water.

What is needed is an easy to use, premeasured cosmetic composition for reducing age-related symptoms in human skin. It would be preferable if it could be delivered in a bag comprising water dispersible paper. It would be further preferable if the cosmetic composition included an oil which would form an emulsion in hot water if agitated. It would be further preferable if the cosmetic composition included at least one effervescent compound that would function as the agitator for emulsifying the oil

### SUMMARY

The present technology is a premeasured cosmetic composition for reducing age-related symptoms in human skin. It is delivered in a bag comprising water dispersible paper. The cosmetic composition includes an oil which forms an emulsion in hot water if agitated. The cosmetic composition includes at least one effervescent compound that functions as the agitator for emulsifying the oil.

In one embodiment, a cosmetic sachet is provided for reducing age-related symptoms in human skin, the cosmetic sachet comprising: a bag comprising a water dispersing paper; and a cosmetic composition, the cosmetic composition releasably retained in the bag and comprising sodium bicarbonate, citric acid, magnesium sulfate heptahydrate, starch, highly saturated plant oil and methylene blue.

In the cosmetic sachet, the highly saturated plant oil may be one or more of coconut oil and palm kernel oil.

In the cosmetic sachet, the starch may be selected from the group consisting of corn starch, rice starch, arrowroot starch, tapioca starch and mixtures thereof.

In the cosmetic sachet, the cosmetic composition may comprise 32% to 41.5% sodium bicarbonate, 17% to 18.6% citric acid, 17% to 18.6% magnesium sulfate heptahydrate, 17% to 18.6% starch, 6% to 13% plant oil, which is at least 80% saturated and 0.036% to 0.04% methylene blue.

In the cosmetic sachet, the cosmetic composition may comprise 38% sodium bicarbonate, 18.6% citric acid, 18.6% magnesium sulfate heptahydrate, 18.6% starch, 6% plant oil and 0.04% methylene blue.

In the cosmetic sachet, the starch may be selected from the group consisting of corn starch, rice starch, arrowroot starch, tapioca starch and mixtures thereof.

In the cosmetic sachet, the highly saturated plant oil may be one or more of coconut oil and palm kernel oil.

In the cosmetic sachet, the highly saturated plant oil may be coconut oil.

In the cosmetic sachet, the starch may be corn starch.

In another embodiment, a method of preparing a topical formulation is provided for reducing age-related symptoms in human skin, the method comprising:
a.—selecting a cosmetic sachet, the cosmetic sachet comprising: a bag comprising a water dispersing paper; and a cosmetic composition, the cosmetic composition releasably retained in the bag and comprising sodium bicarbonate, citric acid, magnesium sulfate heptahydrate, starch, highly saturated plant oil and methylene blue;
b.—and delivering the cosmetic sachet to a volume of hot water such that the final concentration of methylene blue is one micromolar, thereby preparing the topical formulation.

In the method, the cosmetic composition may comprise 32% to 41.5% sodium bicarbonate, 17% to 18.6% citric acid, 17% to 18.6% magnesium sulfate heptahydrate, 17% to 18.6% starch, 6% to 13% plant oil, which is at least 80% saturated and 0.036% to 0.04% methylene blue.

In the method, the cosmetic composition may comprise 38% sodium bicarbonate, 18.6% citric acid, 18.6% magnesium sulfate heptahydrate, 18.6% starch, 6% plant oil, which is at least 80% saturated and 0.04% methylene blue.

In another embodiment, a topical formulation is provided for reducing age-related symptoms in human skin, the topical formulation comprising: a cosmetic composition which comprises sodium bicarbonate, citric acid, magnesium sulfate heptahydrate, starch, highly saturated plant oil and methylene blue; and water, wherein the concentration of methylene blue is one micromolar.

In the topical formulation, the starch may be selected from the group consisting of corn starch, rice starch, arrowroot starch, tapioca starch and mixtures thereof.

In the topical formulation, the highly saturated plant oil may be one or more of coconut oil and palm kernel oil.

In the topical formulation, the highly saturated plant oil may be coconut oil.

In the topical formulation, the starch may be corn starch.

In the topical formulation, the cosmetic composition may comprise 32% to 41.5% sodium bicarbonate, 17% to 18.6% citric acid, 17% to 18.6% magnesium sulfate heptahydrate, 17% to 18.6% corn starch, 6% to 13% coconut oil and 0.036% to 0.04% methylene blue.

In the topical formulation, the cosmetic composition may comprise 38% sodium bicarbonate, 18.6% citric acid, 18.6% magnesium sulfate heptahydrate, 18.6% corn starch, 6% coconut oil, which is at least 80% saturated and 0.04% methylene blue.

### BRIEF DESCRIPTION OF THE DRAWINGS

FIG. 1 is a cross sectional view of the cosmetic sachet of the present technology.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising.” “having.” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Cosmetic composition—in the context of the present technology, a cosmetic composition means a composition formulated for skin care.

Topical formulation—in the context of the present technology, a topical formulation is a formulation for administering to the skin.

Water dispersible paper—in the context of the present technology, water dispersible paper is a material that readily degrades in the presence of water and may include water soluble components.

Highly saturated oil—in the context of the present technology a highly saturated oil is one that is at least about 80% saturated.

### DETAILED DESCRIPTION

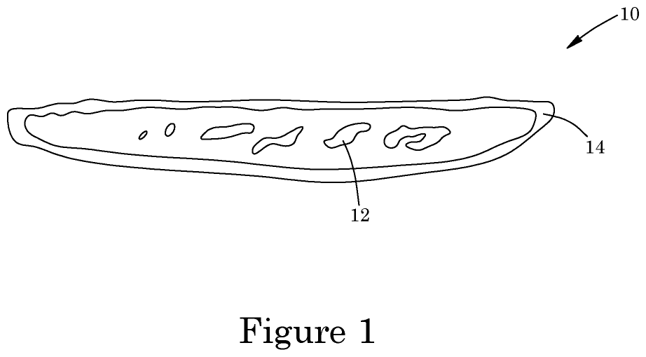

A cosmetic sachet, generally referred to as 10 is shown in FIG. 1. The cosmetic sachet 10 releasably retains a cosmetic composition 12 in a bag 14. The bag 14 is made of a water dispersible paper. The water dispersible paper includes wood pulp and/or non-wood pulp. The water dispersible paper can comprise polyvinyl alcohol (fully- and partially-hydrolyzed polyvinyl acetate), co-polymers based on polyvinyl alcohol (fully- and partially-hydrolyzed vinyl acetate copolymers), cellulose ether, lignin, polymers based on polyvinyl pyrrolidone, polyethylene glycol, xanthan gum, guar gum, polyquaternium polymers, pullulan, hydroxy-propyl-methyl cellulose, hydroxyethyl cellulose, hydroxypropyl cellulose, gelatin, sodium salt of carboxymethylcellulose, sodium alginate, gum tragacanth, acacia gum, gum arabic, polyacrylic acid, methyl-methacrylate copolymer, carboxy-vinyl polymer, amylases, natural and modified starches, aluminum starch octenyl-succinate, hydroxypropyl starch phosphates, high amylase starch, high amylase hydroxy-propylated starch, dextrin, pectin, chitin, chitosan, levan, elsinan, collagen, zein, gluten, soya protein isolate, milk protein isolate, casein, carob bean gum, karaya gum, carrageenan, gellan gum, agar, alginic acid and alginates, furcellaran, polyhydroxy acid polymers, and mixtures thereof.

The water-dispersible materials can make up about 60-100 weight percent of the water dispersible paper, based on the total weight. In addition to the water-dispersible material, other materials such as, but not limited to, plasticizers, stabilizers, pigments, dyes, fillers, and processing aids may be included an amount of about 0-40 weight percent, based on the total weight of the water dispersible paper.

The cosmetic composition 12 per cosmetic sachet 10 is composed of: Sodium bicarbonate from about 5.5 to about 7.5 and preferably 6.5 milliliters; Citric acid from about 2.25 to about 4.25 and preferably 3.25 milliliters; Magnesium sulfate heptahydrate from about 2.25 to about 4.25 and preferably 3.25 milliliters; Starch from about 2.25 to about 4.25 and preferably 3.25 milliliters, wherein the starch is selected from corn starch, rice starch, arrowroot starch, tapioca starch and mixtures thereof; Oil from about 1.0 to about 3, preferably 1.5 milliliters, wherein the oil is selected from coconut oil, palm oil, palm kernel oil and mixtures thereof, in other words, vegetable oils that are highly saturated and are therefore solid at room temperature; and USP/EU standard Methylene blue [7-(dimethylamino)phenothiazine-3-ylidene]-dimethylazanium chloride] from about 0.005 to about 0.011, preferably 0.008 grams or about 0.005 to about 0.011 and preferably about 0.008 milliliters. The cosmetic composition 12 is mixed and then packaged into a bag 14, which is then sealed to form the cosmetic sachet 10. The cosmetic sachet 10 in turn is packaged in a cellophane wrapper to ensure that moisture does not enter the cosmetic sachet 10. In one embodiment, the amount of the cosmetic composition 12 per bag 14 is selected to provide a topical formulation having about 1 μmolar of Methylene Blue in a typical bathtub of roughly 250 liters of water.

In another embodiment, the cosmetic composition 12 per sachet 10 is selected to provide a topical formulation having about 1 μmolar of Methylene Blue in a typical sink of approximately 5 liters of water. The cosmetic composition 12 per cosmetic sachet 10 is composed of: Sodium bicarbonate from about 0.11 to about 0.15 and preferably 0.13 milliliters; Citric acid from about 0.045 to about 0.085 and preferably 0.065 milliliters; Magnesium sulfate heptahydrate from about 0.045 to about 0.085 and preferably 0.065 milliliters; Starch from about 0.045 to about 0.085 and preferably 0.065 milliliters, wherein the starch is selected from corn starch, rice starch, arrowroot starch, tapioca starch and mixtures thereof; Plant oil from about 0.02 to about 0.06, preferably 0.075 milliliters, wherein the plant oil is selected from coconut oil, palm kernel oil and mixtures thereof, in other words, plant oils that are highly saturated (at least about 80% saturation) and are therefore solid at room temperature; and USP/EU standard Methylene blue [7-(dimethylamino)phenothiazine-3-ylidene]-dimethylazanium chloride] from about 5 to about 11, preferably about 8 milligrams or about 5 to about 11 and preferably 8 microliters.

The proportions of components in the cosmetic composition 12 can also be expressed as follows: Sodium bicarbonate from about 32% to about 41.4% and preferably 38.1%; Citric acid from about 17% to about 18.6% and preferably 18%; Magnesium sulfate heptahydrate from about 17% to about 18.6% and preferably 18%; Starch from about 17% to about 18.6% and preferably 18%, wherein the starch is selected from corn starch, rice starch, arrowroot starch, tapioca starch and mixtures thereof; Plant oil from about 6% to about 13%, preferably about 8%, wherein the plant oil is selected from coconut oil, palm kernel oil and mixtures thereof, in other words, plant oils that are highly saturated (at least about 80% saturation) and are therefore solid at room temperature; and USP/EU standard Methylene blue [7-(dimethylamino) phenothiazine-3-ylidene]-dimethylazanium chloride] from about 0.036% to about 0.04%.

In use, a user selects the appropriate size cosmetic sachet 10 for the selected volume of water, then places the cosmetic sachet 10 in that volume of hot water. The bag 14 is dispersed by the hot water, releasing the cosmetic composition 12 creating a topical formulation. The topical formulation is effervescent. The bubbling action emulsifies the plant oil, which is now liquid, and urges the topical formulation throughout the hot water forming a skin soothing wash. The concentration of methylene blue in the hot water is appropriate for decreasing aging related changes to the user's skin.

In an alternative embodiment the sachet is used as a medical treatment for medical conditions relating to aging of the skin including, but not limited to, thinning skin and healing of persistent minor sores and other minor cuts and abrasions. It also promotes the creation of new elastin and collagen, hydrates the skin and significantly reduces lines and wrinkles. The concentration of the active ingredient, methylene blue is the same as described above, as it is relative proportions of the other components in the formulation.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A cosmetic sachet for reducing age-related symptoms in human skin, the cosmetic sachet comprising: a bag comprising a water dispersing paper; and a cosmetic composition, the cosmetic composition releasably retained in the bag and comprising sodium bicarbonate, citric acid, magnesium sulfate heptahydrate, starch, highly saturated plant oil and methylene blue.

2. The cosmetic sachet of claim 1, wherein the highly saturated plant oil is one or more of coconut oil and palm kernel oil.

3. The cosmetic sachet of claim 2, wherein the starch is selected from the group consisting of corn starch, rice starch, arrowroot starch, tapioca starch and mixtures thereof.

4. The cosmetic sachet of claim 1, wherein the cosmetic composition comprises 32% to 41.5% sodium bicarbonate, 17% to 18.6% citric acid, 17% to 18.6% magnesium sulfate heptahydrate, 17% to 18.6% starch, 6% to 13% plant oil, which is at least 80% saturated and 0.036% to 0.04% methylene blue.

5. The cosmetic sachet of claim 4, wherein the cosmetic composition comprises 38% sodium bicarbonate, 18.6% citric acid, 18.6% magnesium sulfate heptahydrate, 18.6% starch, 6% plant oil and 0.04% methylene blue.

6. The cosmetic sachet of claim 5, wherein the starch is selected from the group consisting of corn starch, rice starch, arrowroot starch, tapioca starch and mixtures thereof.

7. The cosmetic sachet of claim 6, wherein the highly saturated plant oil is one or more of coconut oil and palm kernel oil.

8. The cosmetic sachet of claim 7, wherein the highly saturated plant oil is coconut oil.

9. The cosmetic sachet of claim 8, wherein the starch is corn starch.

10. A method of preparing a topical formulation for reducing age-related symptoms in human skin, the method comprising:
selecting a cosmetic sachet, the cosmetic sachet comprising: a bag comprising a water dispersing paper; and a cosmetic composition, the cosmetic composition releasably retained in the bag and comprising sodium bicarbonate, citric acid, magnesium sulfate heptahydrate, starch, highly saturated plant oil and methylene blue;
and delivering the cosmetic sachet to a volume of hot water such that the final concentration of methylene blue is one micromolar, thereby preparing the topical formulation.

11. The method of claim 10, wherein the cosmetic composition comprises 38% sodium bicarbonate, 18.6% citric acid, 18.6% magnesium sulfate heptahydrate, 18.6% starch, 6% plant oil, which is at least 80% saturated and 0.04% methylene blue.

12. A topical formulation for reducing age-related symptoms in human skin, the topical formulation comprising: a cosmetic composition which comprises sodium bicarbonate, citric acid, magnesium sulfate heptahydrate, starch, highly saturated plant oil and methylene blue; and water, wherein the concentration of methylene blue is one micromolar.

13. The topical formulation of claim 12 wherein the starch is selected from the group consisting of corn starch, rice starch, arrowroot starch, tapioca starch and mixtures thereof.

14. The topical formulation of claim 13, wherein the highly saturated plant oil is one or more of coconut oil and palm kernel oil.

15. The topical formulation of claim 14, wherein the highly saturated plant oil is coconut oil.

16. The topical formulation of claim 15 wherein the cosmetic composition comprises 32% to 41.5% sodium bicarbonate, 17% to 18.6% citric acid, 17% to 18.6% magnesium sulfate heptahydrate, 17% to 18.6% corn starch, 6% to 13% coconut oil and 0.036% to 0.04% methylene blue.
